(19) United States
(12) Patent Application Publication
(10) Pub. No.: US 2010/0137456 A1
(43) Pub. Date: Jun. 3, 2010

(54) Polysaccharides functionalized by tryptophan derivatives

(75) Inventors: Rémi Soula, Lyon (FR); Gérard Soula, Meyzieu (FR); Olivier Soula, Meyzieu (FR)

Correspondence Address: OLIFF & BERRIDGE, PLC, P.O. BOX 320850, ALEXANDRIA, VA, 22320-4850, US

(73) Assignee: ADOCIA, LYON (FR)

(21) Appl. No.: 12/461,326

(22) Filed: Aug. 7, 2009

Related U.S. Application Data
(60) Provisional application No. 61/136,121, filed on Aug. 13, 2008.

(30) Foreign Application Priority Data
Aug. 13, 2008 (FR) 08 55567

(51) Int. Cl. A61K 47/36; C08B 37/00; A61P 43/00

(52) U.S. Cl. 514/777; 536/123.1

(57) ABSTRACT

The present invention relates to novel polysaccharide derivatives, predominantly comprising glycosidic bonds of (1,4), (1,3) and/or (1,2) type, functionalized by at least one tryptophan derivative. It also relates to processes for the synthesis thereof, to their uses as pharmaceutical excipient and to the pharmaceutical compositions comprising them.

The present invention relates to novel biocompatible polymers based on polysaccharides.

These polymers can be of use in particular in the administration of active principle(s) (APs) to man or animals for a therapeutic and/or prophylactic purpose.

Polysaccharides, also known as glycans or polyosides, are polymers formed of monosaccharidic units or osides connected via glycosidic bonds. In general, polysaccharides have the general formula [Cₓ(H₂O)ₓ₋₁)]ₙ. These macromolecules are complex because of the variations in size, in branching, in nature of the monosaccharidic unit and in nature of the glycosidic bond.

Two categories of polysaccharides are distinguished:
homopolysaccharides, composed of just one monosaccharidic unit,
heteropolysaccharides, formed of several monosaccharidic units.

Homopolysaccharides are generally distinguished by the nature of the saccharidic unit and the glycosidic bond. The glycosidic bond is the bond formed between the hemiacetal group of a saccharide and the hydroxyl functional group of another saccharide. This bond can be α or β, according to the stereochemistry of the anomeric carbon, but it can in particular be (1,2), (1,3), (1,4) or (1,6), according to the 2, 3, 4 or 6 hydroxyl functional group of the saccharide involved in the bond. Some polysaccharides are composed of the same units but vary because of the bonds involved. For example, dextran and pullulan are both polysaccharides composed of glucose units but, in the case of dextran, the glycosidic bonds are more than 95% (1,6) whereas, in the case of pullulan, they are 67% (1,4) and 33% (1,6). These differences in structure result in differences in physicochemical properties, such as the solubility in organic solvents, the solubility in water or the viscosity.

Numerous examples have been reported among amphiphilic polysaccharides.

Biodex, in patent U.S. Pat. No. 6,646,120, has described carboxymethyldextrans modified by benzylamine. This polysaccharide is predominantly composed of glycosidic units connected via a 1,6 bond. This sequence results in highly fluid polymer solutions being obtained.

Patent FR 0 702 316 of the Applicant Company describes dextrans modified by hydrophobic amino acids, including tryptophan. As above, the dextran is predominantly composed of 1,6 sequences of glycosidic units.

However, dextran is an unusual polysaccharide as it is the only polyoside composed to more than 95% of (1,6) bonds, which confers on it a very good solubility in water, a low viscosity in water and also a good solubility in polar organic solvents, such as dimethyl sulfoxide DMSO.

Polysaccharides can be used as vehicles or excipients in pharmaceutical formulations. For some formulations, the low viscosity of dextran and its high solubility in water may exhibit disadvantages, such as excessively great diffusion from the site of administration or excessively rapid dilution by biological fluids.

Other polysaccharides, such as hyaluronans or alginates, exhibit different physical properties. Hyaluronan derivatives modified by C₁₂ or C₁₈ fatty alkyl chains are described in particular in patent FR 2 794 763. Alginate derivatives modified by fatty alkyl chains are also described in this document.

The studies by Akiyoski et al. (J. Controlled Release, 1998, 54, 313-320) describe pullulans modified by cholesterol. Nevertheless, while the polysaccharides used have a viscosity greater than that of dextran, the grafted hydrophobic groups do not exhibit a satisfactory affinity with some active principles, such as proteins, when they are used as vehicles in pharmaceutical compositions.

The present invention relates to novel polysaccharide derivatives, predominantly comprising glycoside bonds of (1,4), (1,3) and/or (1,2) type, functionalized by at least one tryptophan derivative. These novel amphiphilic polysaccharides have a biocompatibility comparable to dextran derivatives but their viscosity is greater and makes it possible to obtain vehicles for pharmaceutical compositions exhibiting a viscosity sufficient to prevent diffusion from the site of administration. Nevertheless, their hydrophobicity can be easily adjusted without detrimentally affecting their biocompatibility. The use as hydrophobic groups of tryptophan derivatives also makes it possible to obtain good interaction with active principles, in particular by the formation of complexes, which makes it possible to adjust their immobilization.

The polysaccharides according to the invention are predominantly composed of glycosidic bonds of (1,4) and/or (1,3) and/or (1,2) type. They may be neutral, that is to say may not carry acid functional groups, or may be anionic and carry acid functional groups.

They are functionalized by at least one tryptophan derivative, denoted Trp:
said tryptophan derivative being grafted or bonded to the polysaccharides by coupling with an acid functional group, it being possible for said acid functional group to be an acid functional group of an anionic polysaccharide and/or an acid functional group carried by a connecting arm R connected to the polysaccharide via a functional group F, said functional group F resulting from the coupling between the connecting arm R and an —OH functional group of the neutral or anionic polysaccharide,
   F being either an ester, thioester, amide, carbonate, carbamate, ether, thioether or amine functional group,
   R being an optionally branched and/or unsaturated chain comprising between 1 and 18 carbons, comprising one or more heteroatoms, such as O, N and/or S, and having at least one acid functional group,
Trp being a residue of an L and/or D tryptophan derivative, a product of the coupling between the amine of the tryptophan and the at least one acid carried by the R group and/or an acid carried by the anionic polysaccharide.

According to the invention, the functionalized polysaccharides can correspond to the following general formula:

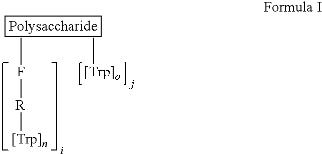

the polysaccharide being predominantly composed of glycoside bonds of (1,4) and/or (1,3) and/or (1,2) type,
F resulting from the coupling between the connecting arm R and an —OH functional group of the neutral or anionic polysaccharide, being either an ester, thioester, amide, carbonate, carbamate, ether, thioether or amine functional group,
R being an optionally branched and/or unsaturated chain comprising between 1 and 18 carbons, comprising one or more heteroatoms, such as O, N and/or S, and having at least one acid functional group,
Trp being a residue of an L and/or D tryptophan derivative, a product of the coupling between the amine of the tryptophan derivative and at least one acid carried by the R group and/or an acid carried by the anionic polysaccharide,
   n representing the molar fraction of the R groups substituted by Trp and being between 0.05 and 0.7,
   o representing the molar fraction of the acid functional groups of the polysaccharides substituted by Trp and being between 0.05 and 0.7,
   i representing the molar fraction of acid functional groups carried by the R group per saccharidic unit and being between 0 and 2,
   j representing the molar fraction of acid functional groups carried by the anionic polysaccharide per saccharidic unit and being between 0 and 1,
   (i+j) representing the molar fraction of acid functional groups per saccharidic unit and being between 0.1 and 2,
   when R is not substituted by Trp, the acid or acids of the R group then being carboxylates of a cation, preferably of an alkali metal, such as Na or K,
   when the polysaccharide is an anionic polysaccharide, when one or more acid functional groups of the polysaccharide are not substituted by Trp, they then being salified by a cation, preferably of an alkali metal, such as Na or K,
said polysaccharides being amphiphilic at neutral pH.

In one embodiment, F is either an ester, a carbonate, a carbamate or an ether.

In one embodiment, the polysaccharide is predominantly composed of glycosidic bonds of (1,4) type.

In one embodiment, the polysaccharide predominantly composed of glycosidic bonds of (1,4) type is chosen from the group consisting of pullulan, alginate, hyaluronan, xylan, galacturonan or a water-soluble cellulose.

In one embodiment, the polysaccharide is a pullulan.

In one embodiment, the polysaccharide is an alginate.

In one embodiment, the polysaccharide is a hyaluronan.

In one embodiment, the polysaccharide is a xylan.

In one embodiment, the polysaccharide is a galacturonan.

In one embodiment, the polysaccharide is a water-soluble cellulose.

In one embodiment, the polysaccharide is predominantly composed of glycosidic bonds of (1,3) type.

In one embodiment, the polysaccharide predominantly composed of glycosidic bonds of (1,3) type is a curdlan.

In one embodiment, the polysaccharide is predominantly composed of glycosidic bonds of (1,2) type.

In one embodiment, the polysaccharide predominantly composed of glycosidic bonds of (1,2) type is an inulin.

In one embodiment, the polysaccharide is predominantly composed of glycosidic bonds of (1,4) and (1,3) type.

In one embodiment, the polysaccharide predominantly composed of glycosidic bonds of (1,4) and (1,3) type is a glucan.

In one embodiment, the polysaccharide is predominantly composed of glycosidic bonds of (1,4) and (1,3) and (1,2) type.

In one embodiment, the polysaccharide predominantly composed of glycosidic bonds of (1,4) and (1,3) and (1,2) type is mannan.

In one embodiment, the polysaccharide according to the invention is characterized in that the R group is chosen from the following groups:

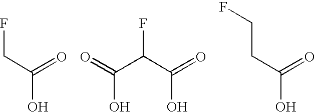

or their salts of alkali metal cations.

In one embodiment, the polysaccharide according to the invention is characterized in that the tryptophan derivative is chosen from the group consisting of tryptophan, tryptophanol, tryptophanamide, 2-indolylethylamine and their alkali metal cation salts.

In one embodiment, the polysaccharide according to the invention is characterized in that the tryptophan derivative is chosen from the tryptophan esters of formula II:

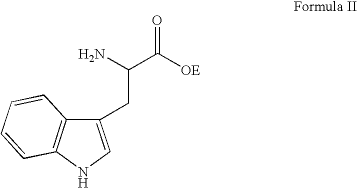

E being a group which can be:
a linear or branched C₁ to C₈ alkyl,
a linear or branched C₆ to C₂₀ alkylaryl or arylalkyl.

The polysaccharide can have a degree of polymerization m of between 10 and 10 000.

In one embodiment, it has a degree of polymerization m of between 10 and 1000.

In another embodiment, it has a degree of polymerization m of between 10 and 500.

In one embodiment, the functionalized polysaccharides are pullulans which correspond to the following general formula III:

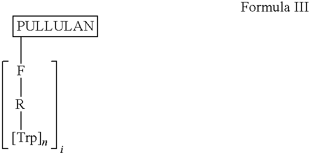

F resulting from the coupling between the connecting arm R and an —OH functional group of a glucose unit, being either an ester, thioester, amide, carbonate, carbamate, ether, thioether or amine functional group,
R being an optionally branched and/or unsaturated chain comprising between 1 and 18 carbons, comprising one or more heteroatoms, such as O, N and/or S, and having at least one acid functional group,
Trp being a residue of an L and/or D tryptophan derivative, a product of the coupling between the amine of the tryptophan derivative and the at least one acid carried by the R group,
   n representing the molar fraction of the R groups substituted by Trp and being between 0.05 and 0.7,
   i representing the molar fraction of acid functional groups carried by the R group per saccharidic unit and being between 0 and 2,
   when R is not substituted by Trp, the acid or acids of the R group then being carboxylates of a cation, preferably of an alkali metal, such as Na or K,
said pullulans being amphiphilic at neutral pH.

In one embodiment, F is either an ester, a carbonate, a carbamate or an ether.

In one embodiment, the pullulan according to the invention is characterized in that the R group is chosen from the following groups:

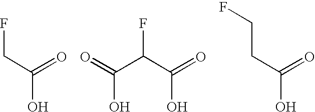

or their salts of alkali metal cations.

In one embodiment, the pullulan according to the invention is characterized in that the tryptophan derivative is chosen from the group consisting of tryptophan, tryptophanol, tryptophanamide, 2-indolylethylamine and their alkali metal cation salts.

In one embodiment, the pullulan according to the invention is characterized in that the tryptophan derivative is chosen from the tryptophan esters of formula II:

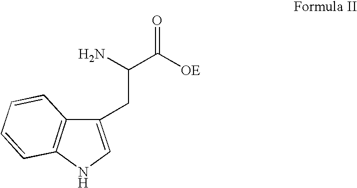

E being a group which can be:
a linear or branched C₁ to C₈ alkyl,
a linear or branched C₆ to C₂₀ alkylaryl or arylalkyl.

The pullulan can have a degree of polymerization m of between 10 and 10 000.

In one embodiment, it has a degree of polymerization m of between 10 and 1000.

In another embodiment, it has a degree of polymerization m of between 10 and 500.

According to the invention, the functionalized polysaccharides are galacturonans which correspond to the following general formula:

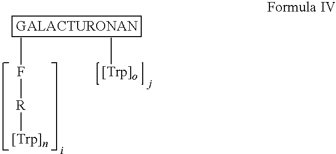

F resulting from the coupling between the connecting arm R and an —OH functional group of the galacturonan, being either an ester, thioester, amide, carbonate, carbamate, ether, thioether or amine functional group,
R being an optionally branched and/or unsaturated chain comprising between 1 and 18 carbons, comprising one or more heteroatoms, such as O, N and/or S, and having at least one acid functional group,
Trp being a residue of an L and/or D tryptophan derivative, a product of the coupling between the amine of the tryptophan derivative and the at least one acid carried by the R group and/or an acid carried by the galacturonan,
   n representing the molar fraction of the R groups substituted by Trp and being between 0.05 and 0.7,
   o representing the molar fraction of the acid functional groups of the galacturonans substituted by Trp and being between 0.05 and 0.7,
   i representing the molar fraction of acid functional groups carried by the R group per saccharidic unit and being between 0 and 2,
   j representing the molar fraction of acid functional groups carried by the galacturonan per saccharidic unit and being between 0 and 1,
   (i+j) representing the molar fraction of acid functional groups per saccharidic unit and being between 0.1 and 2,
   when R is not substituted by Trp, the acid or acids of the R group then being carboxylates of a cation, preferably of an alkali metal, such as Na or K,
   when one or more acid functional groups of the galacturonan are not substituted by Trp, they then being salified by a cation, preferably of an alkali metal, such as Na or K,
said galacturonans being amphiphilic at neutral pH.

In one embodiment, F is either an ester, a carbonate, a carbamate or an ether.

In one embodiment, the galacturonan according to the invention is characterized in that the R group is chosen from the following groups:

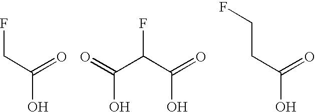

or their salts of alkali metal cations.

In one embodiment, the galacturonan according to the invention is characterized in that the tryptophan derivative is chosen from the group consisting of tryptophan, tryptophanol, tryptophanamide, 2-indolylethylamine and their alkali metal cation salts.

In one embodiment, the galacturonan according to the invention is characterized in that the tryptophan derivative is chosen from the tryptophan esters of formula II:

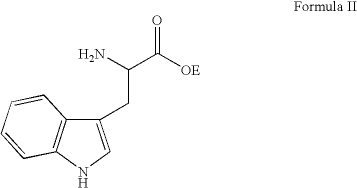

E being a group which can be:
a linear or branched C₁ to C₈ alkyl,
a linear or branched C₆ to C₂₀ alkylaryl or arylalkyl.

The galacturonan can have a degree of polymerization m of between 10 and 10 000.

In one embodiment, it has a degree of polymerization m of between 10 and 1000.

In another embodiment, it has a degree of polymerization m of between 10 and 500.

According to the invention, the functionalized polysaccharides are alginates which correspond to the following general formula:

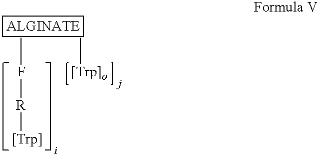

F resulting from the coupling between the connecting arm R and an —OH functional group of the alginate, being either an ester, thioester, amide, carbonate, carbamate, ether, thioether or amine functional group,
R being an optionally branched and/or unsaturated chain comprising between 1 and 18 carbons, comprising one or more heteroatoms, such as O, N and/or S, and having at least one acid functional group,
Trp being a residue of an L and/or D tryptophan derivative, a product of the coupling between the amine of the tryptophan derivative and the at least one acid carried by the R group and/or an acid carried by the alginate,
   n representing the molar fraction of the R groups substituted by Trp and being between 0.05 and 0.7,
   o representing the molar fraction of the acid functional groups of the alginates substituted by Trp and being between 0.05 and 0.7,
   i representing the molar fraction of acid functional groups carried by the R group per saccharidic unit and being between 0 and 2,
   j representing the molar fraction of acid functional groups carried by the alginate per saccharidic unit and being between 0 and 1,
   (i+j) representing the molar fraction of acid functional groups per saccharidic unit and being between 0.1 and 2,
   when R is not substituted by Trp, the acid or acids of the R group then being carboxylates of a cation, preferably of an alkali metal, such as Na or K,
   when one or more acid functional groups of the polysaccharide are not substituted by Trp, they then being salified by a cation, preferably of an alkali metal, such as Na or K,
said alginates being amphiphilic at neutral pH.

In one embodiment, F is either an ester, a carbonate, a carbamate or an ether.

In one embodiment, the alginate according to the invention is characterized in that the R group is chosen from the following groups:

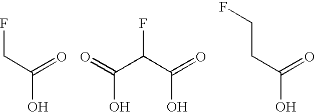

or their salts of alkali metal cations.

In one embodiment, the alginate according to the invention is characterized in that the tryptophan derivative is chosen from the group consisting of tryptophan, tryptophanol, tryptophanamide, 2-indolylethylamine and their alkali metal cation salts.

In one embodiment, the alginate according to the invention is characterized in that the tryptophan derivative is chosen from the tryptophan esters of formula II

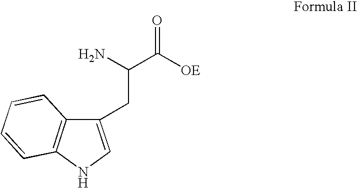

E being a group which can be:
a linear or branched C₁ to C₈ alkyl,
a linear or branched C₆ to C₂₀ alkylaryl or arylalkyl.

The alginate can have a degree of polymerization m of between 10 and 10 000.

In one embodiment, it has a degree of polymerization m of between 10 and 1000.

In another embodiment, it has a degree of polymerization m of between 10 and 500.

In another embodiment, the polysaccharides according to the invention are obtained by grafting a tryptophan derivative as defined above to a neutral polysaccharide, by coupling between the amine functional group of the tryptophan derivative and an acid functional group obtained by grafting an R group carrying at least one acid functional group as defined above to an alcohol functional group of the polysaccharide, in order to obtain polysaccharides of formula I in which j=0.

In one embodiment, the polysaccharides according to the invention are obtained by grafting a tryptophan derivative as defined above to an acid functional group of an anionic polysaccharide, by coupling between the amine functional group of the tryptophan derivative and an acid functional group carried by the anionic polysaccharide, in order to obtain polysaccharides of formula I in which i=0.

In one embodiment, when the polysaccharide is an anionic polysaccharide, R groups can be grafted to the alcohol functional groups of the polysaccharide and the grafting of the tryptophan derivative can be carried out:
either selectively on the acid functional groups of the R groups, by protection/deprotection reactions well known to a person skilled in the art, in order to obtain polysaccharides of formula I in which o=0, or
jointly on both types of acid functional groups, in order to obtain polysaccharides of formula I in which n>0 and o>0.

In all the embodiments described above, the coupling reactions are followed by the neutralization of the acid functional groups which are not reacted with a tryptophan derivative by salification by one of the methods well known to a person skilled in the art, in order to obtain a salt of an alkali metal cation, preferably Na or K.

The invention also relates to a pharmaceutical composition comprising one of the polysaccharides according to the invention as described above and at least one active principle.

Active principle is understood to mean a product in the form of a single chemical entity or in the form of a combination having a physiological activity. Said active principle can be exogenous, that is to say that it is introduced by the composition according to the invention. It can also be endogenous, for example growth factors, which will be secreted in a wound during the first phase of healing and which can be retained on said wound by the composition according to the invention.

The invention also relates to a pharmaceutical composition according to the invention as described above, characterized in that it can be administered orally, nasally, vaginally or buccally.

The invention also relates to a pharmaceutical composition according to the invention as described above, characterized in that it is obtained by drying and/or lyophilization.

The invention also relates to a pharmaceutical composition according to the invention as described above, characterized in that it can be administered in the form of a stent, film or coating of implantable biomaterials, or implant.

The invention also relates to a pharmaceutical composition according to the invention as described above, characterized in that the active principle is chosen from the group consisting of proteins, glycoproteins, peptides and nonpeptide therapeutic molecules.

The pharmaceutical compositions possible are either in the liquid form (nanoparticles or microparticles in suspension in water or in mixtures of solvents) or in the powder, implant or film form.

In the case of local and systemic releases, the methods of administration envisaged are intravenously, subcutaneously, intradermally, intramuscularly, orally, nasally, vaginally, ocularly, buccally, and the like.

The invention also relates to the use of the functionalized polysaccharides according to the invention in the preparation of pharmaceutical compositions as described above.

The invention is illustrated by the following examples.

### EXAMPLE 1

### Synthesis of a Sodium Pullulanmethylcarboxylate Modified by the Sodium Salt of Tryptophan, Polymer 1

8 g (i.e., 148 mmol of hydroxyl functional groups) of pullulan with a weight-average molar mass of approximately 100 kg/mol (Fluka) are dissolved in water at 42 g/l. 15 ml of 10N NaOH (148 mmol of NaOH) are added to this solution. The mixture is brought to 35° C. and then 23 g (198 mmol) of sodium chloroacetate are added. The temperature of the reaction medium is brought to 60° C. at 0.5° C./min and then maintained at 60° C. for 100 minutes. The reaction medium is diluted with 200 ml of water, neutralized with acetic acid and purified by ultrafiltration through a 5 kD PES membrane against 6 volumes of water. The final solution is assayed by solids dry content, to determine the concentration of polymer, and then assayed by acid/base titration in H₂O/acetone 50/50 (V/V), to determine the degree of substitution with carboxymethylate.

From the solids dry content: [polymer]=31.5 mg/g

From the acid/base titration: the degree of substitution of the hydroxyl functional groups by methylcarboxylate functional groups is 1.17 per saccharidic unit.

The sodium pullulanmethylcarboxylate solution is passed over a Purolite (anionic) resin in order to obtain pullulanmethylcarboxylic acid, which is subsequently lyophilized for 18 hours.

3.51 g of pullulanmethylcarboxylic acid (i.e., 18 mmol of carboxymethyl acidic functional groups) are dissolved in DMF at 57 g/l and then cooled to 0° C. 1.81 g (18 mmol) of NMM and 1.94 g (18 mmol) of EtOCOCl are subsequently added. After reacting for 10 min, 1.40 g (7 mmol) of TrpOH are added. The medium is subsequently heated to 10° C. and maintained at this temperature for 30 minutes. A 340 g/l solution of imidazole (2.43 g, 36 mmol) in water is subsequently added and the reaction medium is briefly heated at 30° C. The reaction medium is subsequently diluted with 70 ml of water and then filtered through a sintered glass funnel, porosity 1, and then through a sintered glass funnel, porosity 3. It is then clear. The solution is ultrafiltered through a 10 kD PES membrane against 10 volumes of 0.9% NaCl solution and then 6 volumes of water. The concentration of the polymer solution is determined by solids content. A fraction of solution is lyophilized and analyzed by ¹H NMR in D₂O in order to determine the DS with grafted tryptophan.

From the ¹H NMR: the molar fraction of the acids modified by the tryptophan is 0.4.

### EXAMPLE 2

### Synthesis of a Sodium Pullulan Succinic Carboxylate Modified by the Sodium Salt of Tryptophan

10 g of pullulan with a weight-average molar mass of approximately 100 000 g/mol (Fluka) are dissolved in DMSO at 400 mg/g at 60° C. This solution is heated to 40° C. and then two solutions of 9.27 g of succinic anhydride (371 mg/ml in DMF) and of 9.37 g of NMM (375 mg/ml in DMF) are added to the polymer solution. The reaction time is 240 min starting from the addition of the NMM solution. The solution thus obtained is diluted with 1 l of water and ultrafiltered through a 10 kD PES membrane. The final solution is assayed by solids dry content, in order to determine the concentration of polymer, and then assayed by ¹H NMR in D₂O NaOD, in order to determine the DS with grafted succinate.

From the solids dry content: [polymer]=15.8 mg/g

From the ¹H NMR: the molar fraction of the alcohols modified by sodium succinate is 1.35.

The sodium pullulan succinic carboxylate solution is passed over a Purolite (anionic) resin in order to obtain the pullulan succinic carboxylic acid, which is subsequently lyophilized for 18 hours.

5.88 g of pullulan succinic carboxylic acid (i.e., 27 mmol of SA functional groups) are dissolved in DMF at 45 g/l and then cooled to 0° C. 0.90 g (8.9 mmol) of NMM and 0.97 g (8.9 mmol) of EtOCOCl are subsequently added. After reacting for 10 min, 5.46 g (27 mmol) of TrpOH are added. The medium is subsequently heated to 30° C. and maintained at this temperature for 3 hours. A 340 g/l solution of imidazole (1.82 g, 27 mmol) in water is subsequently added. The reaction medium is subsequently diluted with 75 ml of water; it is then clear. The solution is purified by dialysis through an 8 kD regenerated cellulose membrane in 3 times 8 liters of 0.9% NaCl solution and 2 times 8 liters of water. The purified solution is completely lyophilized. The lyophilisate is analyzed by ¹H NMR in D₂O NaOD in order to determine the DS with grafted tryptophan.

From the ¹H NMR: the molar fraction of the acids modified by the tryptophan is 0.4.

### EXAMPLE 3

### Synthesis of Sodium Alginate Modified by Sodium Tryptophan

5 g (25 mmol of carboxylate functional groups) of sodium alginate (Fluka 71238) are dissolved (50 mmol/l as carboxylate functional groups) in a 0.001N aqueous HCl solution. The solution obtained is cooled to 4° C. and the pH is lowered to 4 by addition of 1N HCl. 4.84 g (25 mmol) of N-(3-dimethylaminopropyl)-N′-ethylcarbodiimide hydrochloride (EDC, Fluka 03450) are then added. When the pH of the mixture has stabilized, 13.6 g (50 mmol) of tryptophan ethyl ester hydrochloride (TrpOEt.HCl, Bachem E-2510) are added. After stirring at 4° C. for 30 minutes, the reaction medium is brought to 25° C. and stirring is maintained for 24 hours. The reaction medium is subsequently diluted in a sodium hydroxide solution such that the pH of the mixture is greater than 12. The mixture, which has become clear, is purified by dialysis through an 8 kD membrane against a 0.9% NaCl solution and then against water. The purified polymer solution is finally lyophilized.

The lyophilisate is analyzed by ¹H NMR in D₂O NaOD in order to determine the degree of substitution DS with grafted tryptophan per saccharide unit. From the ¹H NMR, the DS with tryptophan per saccharide unit is 0.25. The distribution of the molar masses of the final polymer is analyzed by Steric Exclusion Chromatography. The chromatogram makes it possible to validate the absence of secondary reaction, such as the coupling of chains or the cleaving of chains.

### EXAMPLE 4

### Synthesis of Galacturonate Modified by Sodium Tryptophan

4.8 g (25 mmol of carboxylate functional groups) of pectin (91% RCOONa, 9% RCOOMe, SigmaAldrich P9135) are dissolved (50 mmol/l as carboxylate functional groups) in a 0.001N aqueous HCl solution. The solution obtained is cooled to 4° C. 4.72 g (25 mmol) of N-(3-dimethylaminopropyl)-N′-ethylcarbodiimide hydrochloride (EDC, Fluka 03450) are then added. When the pH of the mixture has stabilized, 13.2 g (50 mmol) of tryptophan ethyl ester hydrochloride (TrpOEt.HCl, Bachem E-2510) are added. After stirring at 4° C. for 30 minutes, the reaction medium is brought to 25° C. and stirring is maintained for 24 hours. The reaction medium is subsequently diluted in a sodium hydroxide solution such that the pH of the mixture is greater than 12. The mixture, which has become clear, is purified by ultrafiltration through a 50 kD cutoff threshold membrane against 9 volumes of water and concentrated. A solution with a solids dry content of 17 mg/g is obtained.

The DS with tryptophan per saccharidic unit is 0.15, from assaying by UV spectrometry.

A fraction of the solution is lyophilized and then analyzed by ¹H NMR in D₂O. From this analysis, the DS with tryptophan per saccharidic unit is approximately 0.17.

### EXAMPLE 5

### Preparation of a Sodium Dextranmethylcarboxylate Functionalized by Tryptophan

This polymer is a comparative example.

Polymer 5 is a sodium dextranmethylcarboxylate modified by the sodium salt of L-tryptophan obtained from a dextran with a weight-average molar mass of 40 kg/mol (Pharmacosmos) according to the process described in patent application FR07.02316. The molar fraction of sodium methylcarboxylate, modified or not modified by tryptophan, per saccharide unit is 1.03. The molar fraction of sodium methylcarboxylates modified by tryptophan per saccharidic unit is 0.36.

### COUNTEREXAMPLE 1

### Synthesis of a Sodium Pullulanmethyl-carboxylate, Polymer 6

This polymer is obtained according to the process described in the first part of example 1. The stages of acidification and of grafting with tryptophan are not carried out.

The degree of substitution of the hydroxyl functional groups by methylcarboxylate functional groups is 1.17 per saccharidic unit. This polymer is used as counterexample to this invention.

### EXAMPLE 6

### Demonstration of the Affinity of a Polymer for a Protein Which Binds to Heparin by Coelectrophoresis

Preparation of the Protein/polymer Complex in the Ratio 1/500

1.5 μg of protein are added to 750 μg of polymer and to 15 μl of 10× migration buffer (Tris-acetate pH 7). The solution is made up to 150 μl with H₂O. This solution is incubated at ambient temperature for 20 minutes. 5 μl of this second solution containing 50 ng of protein and 25 μg of polymer are diluted in 5 μl of 1× migration buffer. Similar solutions containing only the protein or the polymer are prepared as controls.

Demonstration of the complex between the protein and the polymer

The protein/polymer solution (10 μl) is mixed with 3 μl of loading buffer (glycerol, Tris-acetate and bromophenol blue in water). These 13 μl, containing 50 ng of protein and 25 μg of polymer, are deposited in a well of a 0.8% agarose gel. The control solutions (protein alone or polymer alone) are deposited in a similar fashion. The electrophoresis tank is closed and the generator is adjusted to 30V. Migration lasts 1 hour.

After migration, the gel is transferred onto a PVDF membrane by capillary action with an Apelex system for 2 h at ambient temperature. The membrane is subsequently saturated with skimmed milk for 1 hour at ambient temperature, then incubated with rabbit primary antibodies directed against the protein (overnight at 4° C.) and, finally, incubated with secondary antibodies, rabbit anti-goat HRP (1 hour at ambient temperature). Visualization takes place by reaction of the HRP with Opti-4CN. Visualization is stopped by rinsing in water when the coloration is sufficient since the reaction product absorbs in the visible region.

When the protein is alone or does not form a complex with the polymer, it can migrate, if it is anionic, or can remain at the point of the deposition, if it is cationic. The protein is then detected either at the loading wells or in the form of a single spot at approximately 0.3-0.4 cm from the deposition. When the protein forms a complex with the polymer, the complex is carried along more strongly by the charges of the polymer and moves toward the anode. It is detected in the form of a single spot at 0.7 cm from the deposition. The intensity of this spot varies according to the amount of protein carried along by the polymer. The analysis is regarded as semiquantitative since there is a relationship between the intensity of the spot and the scale of the affinity. Thus, the affinity of a polymer for a protein is denoted “−” when there is no spot detected at 0.7 cm from the deposition, “+” when there is a visible spot of moderate intensity at 0.7 cm from the deposition and “++” when this spot at 0.7 cm from the deposition has a very strong intensity demonstrating a high affinity.

The results obtained with polymer 1, obtained in example 1, polymer 5, obtained in example 5, and proteins chosen from the groups of cell adhesion molecules, coagulation proteins, heparin-binding growth factors, growth factor binding proteins, cytokines and lipid metabolism proteins are collated in table I below.

TABLE I

| | | Polymers | | |
|---|---|---|---|---|
| | | Polymer 1 | Polymer 5 | |
| | | (pullulanmethyl- | (dextranmethyl- | |
| | | carboxylate | carboxylate | Polymer 6 |
| Protein | | substituted by | substituted by | (pullulanmethyl- |
| family | Protein | tryptophan) | tryptophan) | carboxylate) |
| Cell | Pecam-1 | ++ | + | − |
| adhesion | (CD31) | | | |
| molecules | | | | |
| Coagulation | Tissue | ++ | + | − |
| protein | plasminogen | | | |
| | activator (tPa) | | | |
| Growth | IGF-BP-3 | ++ | + | − |
| factor | | | | |
| binding | | | | |
| protein | | | | |
| Cytokine | Interferon- | ++ | + | − |
| | gamma | | | |
| | C-C motif | ++ | + | − |
| | chemokine 1 | | | |
| Lipid | Apo-E | ++ | + | − |
| metabolism | | | | |
| proteins | | | | |
| Heparin- | PDGF-BB | + | ++ | − |
| binding | | | | |
| growth | | | | |
| factors | | | | |

The results obtained show that the grafting of tryptophan to a polysaccharide, such as pullulanmethylcarboxylate, makes it possible to confer, on this polymer, a property of interaction with the proteins studied (results with polymer 1) which pullulanmethylcarboxylate does not have (results with polymer 6).

The results obtained show that pullulanmethylcarboxylate substituted with tryptophan, polymer 1 (example 1), has a greater affinity than that of dextranmethylcarboxylate substituted by tryptophan, polymer 5 (example 5), for the first 6 proteins in table I.

On the other hand, this improvement in the affinity is not systematic since, in the case of PDGF-BB, for example, the affinity of polymer 5 is greater than that of polymer 1.

### EXAMPLE 7

### Viscosity of Polysaccharides

The viscosity of the precursor polysaccharides were studied using a TA AR2000ex rheometer.

The pullulan precursor of polymer 1 has a viscosity of 14 mPa.s at a concentration of 77 mg/ml.

The dextran precursor of polymer 5 has a viscosity of 15 mPa.s at a concentration of 164 mg/ml.

The pullulan employed is approximately twice as viscous as the dextran employed.

1. Functionalized polysaccharide chosen from the group consisting of the polysaccharides of general formula I: 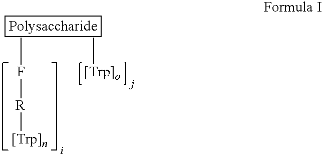
the polysaccharide being predominantly composed of glycosidic bonds of (1,4) and/or (1,3) and/or (1,2) type,
F resulting from the coupling between the connecting arm R and an —OH functional group of the neutral or anionic polysaccharide, being either an ester, thioester, amide, carbonate, carbamate, ether, thioether or amine functional group,
R being an optionally branched and/or unsaturated chain comprising between 1 and 18 carbons, comprising one or more heteroatoms, such as O, N and/or S, and having at least one acid functional group,
Trp being a residue of an L and/or D tryptophan derivative, a product of the coupling between the amine of the tryptophan derivative and the at least one acid carried by the R group and/or an acid carried by the anionic polysaccharide,
n representing the molar fraction of the R groups substituted by Trp and being between 0.05 and 0.7,
representing the molar fraction of the acid functional groups of the polysaccharides substituted by Trp and being between 0.05 and 0.7,
i representing the molar fraction of acid functional groups carried by the R group per saccharidic unit and being between 0 and 2,
j representing the molar fraction of acid functional groups carried by the anionic polysaccharide per saccharidic unit and being between 0 and 1,
(i+j) representing the molar fraction of acid functional groups per saccharide unit and being between 0.1 and 2,
when R is not substituted by Trp, the acid or acids of the R group then being carboxylates of a cation, such as Na or K,
when the polysaccharide is an anionic polysaccharide, when one or more acid functional groups of the polysaccharide are not substituted by Trp, they then being salified by a cation, such as Na or K,
said polysaccharides being amphiphilic at neutral pH.

2. Polysaccharide according to claim 1, wherein F is either an ester, a carbonate, a carbamate or an ether.

3. Polysaccharide according to claim 1, wherein the polysaccharide is predominantly composed of glycosidic bonds of (1,4) type.

4. Polysaccharide according to claim 3, wherein the polysaccharide predominantly composed of glycosidic bonds of (1,4) type is chosen from the group consisting of pullulan, alginate, hyaluronan, xylan, galacturonan or a water-soluble cellulose.

5. Polysaccharide according to claim 1, wherein the polysaccharide is predominantly composed of glycosidic bonds of (1,3) type.

6. Polysaccharide according to claim 5, wherein the polysaccharide predominantly composed of glycosidic bonds of (1,3) type is a curdlan.

7. Polysaccharide according to claim 1, wherein the polysaccharide is predominantly composed of glycosidic bonds of (1,2) type.

8. Polysaccharide according to claim 7, wherein the polysaccharide predominantly composed of glycosidic bonds of (1,2) type is an inulin.

9. Polysaccharide according to claim 1, wherein the polysaccharide is predominantly composed of glycosidic bonds of (1,4) and (1,3) type.

10. Polysaccharide according to claim 9, wherein the polysaccharide predominantly composed of glycosidic bonds of (1,4) and (1,3) type is a glucan.

11. Polysaccharide according to claim 1, wherein the polysaccharide is predominantly composed of glycosidic bonds of (1,4) and (1,3) and (1,2) type.

12. Polysaccharide according to claim 11, wherein the polysaccharide predominantly composed of glycosidic bonds of (1,4) and (1,3) and (1,2) type is mannan.

13. Polysaccharide according to claim 1, wherein the R group is chosen from the following groups: 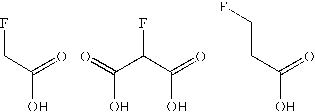
or their salts of alkali metal cations.

14. Polysaccharide according to claim 1, wherein the tryptophan derivative is chosen from the group consisting of tryptophan, tryptophanol, tryptophanamide, 2-indolylethylamine and their alkali metal cation salts.

15. Polysaccharide according to claim 1, wherein the tryptophan derivative is chosen from the tryptophan esters of formula II: 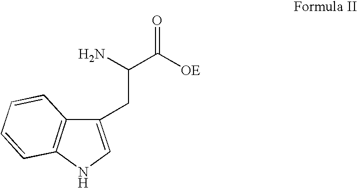
E being a group which can be:
a linear or branched C₁ to C₈ alkyl,
a linear or branched C₆ to C₂₀ alkylaryl or arylalkyl.

16. Pharmaceutical composition, comprising one of the polysaccharides according to claim 1 and at least one active principle.

17. (canceled)

18. A method of preparing a pharmaceutical composition comprising:
providing the functionalized polysaccharide of claim 1.
